(19) United States
(12) Patent Application Publication
(10) Pub. No.: US 2024/0049967 A1
(43) Pub. Date: Feb. 15, 2024

(54) PHYSIOLOGICAL INFORMATION PROCESSING APPARATUS AND PHYSIOLOGICAL INFORMATION PROCESSING METHOD

(71) Applicant: NIHON KOHDEN CORPORATION, Tokyo (JP)

(72) Inventors: Yoshiharu HARADA, Tokorozawa-shi, Saitama (JP); Yoshihiro SUGO, Tokorozawa-shi, Saitama (JP); Sayaka YAMAZAKI, Tokorozawa-shi, Saitama (JP)

(21) Appl. No.: 18/364,858

(22) Filed: Aug. 3, 2023

(30) Foreign Application Priority Data
Aug. 10, 2022 (JP) 2022-127835

(51) Int. Cl. A61B 5/02; A61B 5/021; A61B 5/00

(52) U.S. Cl. CPC A61B 5/02028; A61B 5/02108; A61B 5/7285

(57) ABSTRACT

A physiological information processing apparatus includes a calculator configured to calculate a moving average value of a pulse wave transit time of a subject, and a hemodynamic parameter of the subject, using the calculated moving average value. The calculator is configured to switch a calculation target between a first hemodynamic parameter and a second hemodynamic parameter. The first hemodynamic parameter is calculated using the moving average value of a first pulse wave transit time. The second hemodynamic parameter is calculated using the moving average value of a second pulse wave transit time that is shorter than the moving average time of the first pulse wave transit time.

### CROSS-REFERENCE TO RELATED APPLICATIONS

This application is based on and claims priority under 35 USC 119 from Japanese Patent Application No. 2022-127835 filed on Aug. 10, 2022, the entire content of which is incorporated herein by reference.

### TECHNICAL FIELD

The present disclosure relates to a physiological information processing apparatus and a physiological information processing method.

### BACKGROUND

In the related art, in a case of performing a treatment such as administration of fluid to a subject, a change in a parameter related to hemodynamics (hereinafter, referred to as a “hemodynamic parameter”) such as a stroke volume or a cardiac output of the subject is used as a useful parameter for checking a state of the subject. For example, a small amount of fluid is administered, and it is determined that the administration of fluid is continued in a case where a change rate between a value of the hemodynamic parameter before the administration and a value of the hemodynamic parameter after the administration is equal to or greater than a certain value, and it is determined that the administration is ended to switch to another treatment in a case where the change rate is less than the certain value. Further, JP2005-312947A discloses a method of calculating a cardiac output by using a pulse wave transit time.

A value of the pulse wave transit time used to calculate the hemodynamic parameter may change greatly under an influence of noise or the like. Therefore, for example, the hemodynamic parameter is calculated using a moving average value of a plurality of pulse wave transit times. However, in the above method, since a time corresponding to the plurality of pulse wave transit times is required before the moving average value is calculated, a hemodynamic change cannot be quickly checked.

### SUMMARY

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect, a physiological information processing apparatus including:
a calculator configured to calculate:
   a moving average value of a pulse wave transit time of a subject; and
   a hemodynamic parameter of the subject, using the calculated moving average value,
in which the calculator is configured to switch a calculation target between a first hemodynamic parameter and a second hemodynamic parameter, the first hemodynamic parameter being calculated using the moving average value of a first pulse wave transit time, the second hemodynamic parameter being calculated using the moving average value of a second pulse wave transit time that is shorter than the moving average time of the first pulse wave transit time.

According to an aspect, a physiological information processing method including:
calculating:
   a moving average value of a pulse wave transit time of a subject; and
   a hemodynamic parameter of the subject using the calculated moving average value,
in which, in the calculating, a calculation target is switchable between a first hemodynamic parameter and a second hemodynamic parameter, the first hemodynamic parameter being calculated using the moving average value of a first pulse wave transit time, the second hemodynamic parameter being calculated using the moving average value of a second pulse wave transit time that is shorter than the moving average time of the first pulse wave transit time.

### BRIEF DESCRIPTION OF DRAWINGS

Exemplary embodiment(s) of the presently disclosed subject matter will be described in detail based on the following figures, wherein:

FIG. 1 illustrates a configuration of a physiological information processing apparatus (physiological information display device) according to an aspect of the present disclosure;

FIG. 2 illustrates an example of a measurement mode using a monitor device as an example of the physiological information processing apparatus illustrated in FIG. 1;

FIG. 3 illustrates an example of a screen displayed on a display of FIG. 1;

FIG. 4 illustrates an example of a screen displayed in a case where a treatment for a subject is newly started;

FIG. 5 illustrates an operation for instructing switching of a pulse wave transit time used to calculate a hemodynamic parameter; and

FIG. 6 is a flowchart illustrating an operation of switching the pulse wave transit time used to calculate the hemodynamic parameter, in the physiological information processing apparatus according to the embodiment of the present disclosure.

### DESCRIPTION OF EMBODIMENTS

Exemplary embodiments of a physiological information processing apparatus and a physiological information processing method according to a present disclosure will be described below with reference to the accompanying drawings.

<Configuration of Physiological Information Processing Apparatus>

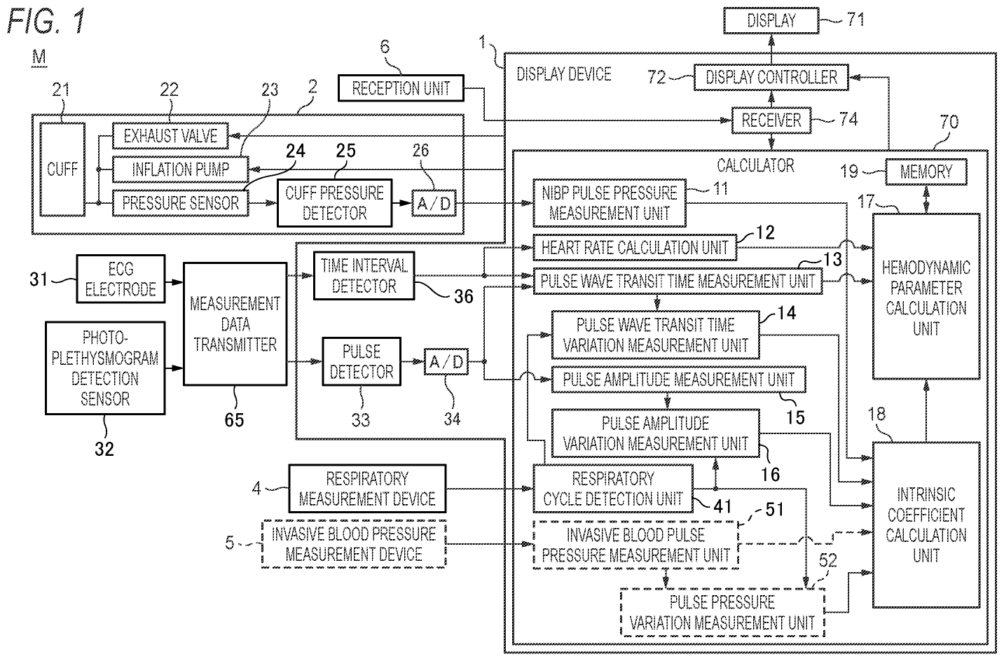

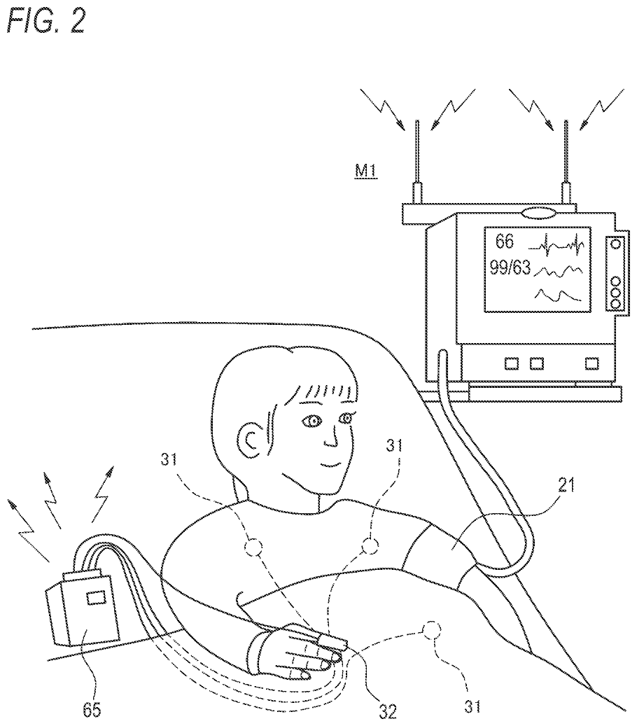

FIG. 1 illustrates a configuration of a physiological information processing apparatus (physiological information display device) M according to an aspect of the presently disclosed subject matter. FIG. 2 illustrates an example of a measurement mode using a monitor device M1, which is an example of the physiological information processing apparatus M of FIG. 1.

The physiological information processing apparatus M can include: a display device 1 configured to perform calculation, display control, and the like of a hemodynamic parameter related to hemodynamics of a subject; a blood pressure measurement device 2 configured to measure a blood pressure in a systolic phase and a diastolic phase of a heart; a respiratory measurement device 4; an invasive blood pressure measurement device 5; a reception unit 6; ECG electrodes 31; a photoplethysmogram detection sensor 32; a measurement data transmitter 65; and a display 71.

The blood pressure measurement device 2 is a device configured to measure a blood pressure of a subject by a non-invasive blood pressure (NIBP) measurement method. The blood pressure measurement device 2 can include a cuff 21, an exhaust valve 22, an inflation pump 23, a pressure sensor 24, a cuff pressure detector 25, and an A/D converter 26. Specifically, as illustrated in FIG. 2, the blood pressure measurement device 2 is configured to measure the blood pressure by attaching the cuff 21 to an upper arm of a subject.

An interior of the cuff 21 is configured to be opened or closed with respect to the atmosphere by opening and closing the exhaust valve 22. The exhaust valve 22 is configured to be opened and closed based on, for example, a control signal received from the monitor device M1. The inflation pump 23 is configured to supply air to the cuff 21. The supply of air is controlled based on, for example, a control signal received from the monitor device M1.

The pressure sensor 24 is connected with the cuff 21, and a sensor output of the pressure sensor 24 is detected by the cuff pressure detector 25. The sensor output from the cuff pressure detector 25 is converted into a digital signal by the A/D converter 26, and then input to an NIBP pulse pressure measurement unit 11 of the display device 1.

As illustrated in FIG. 2, the ECG electrodes 31 are attached to a chest of a subject. The ECG electrodes 31 are configured to perform measurement with an R wave generation time point of an electrocardiogram as a reference point of a time interval. The ECG electrodes 31 are electrically connected to the measurement data transmitter 65. The measurement data obtained by the ECG electrodes 31 is input to the measurement data transmitter 65, and wirelessly transmitted from the measurement data transmitter 65 to a time interval detector 36 in the display device 1 illustrated in FIG. 1.

As illustrated in FIG. 2, the photoplethysmogram detection sensor 32 is attached to the subject's periphery such as a finger, and configured to measure, for example, pulse waves. A pulse wave transit time (PWTT) is obtained from the measurement data obtained from the ECG electrodes 31, and the pulse waves obtained from the photoplethysmogram detection sensor 32. The photoplethysmogram detection sensor 32 is electrically connected to the measurement data transmitter 65. The measurement data obtained by the photoplethysmogram detection sensor 32 is input to the measurement data transmitter 65, and is wirelessly transmitted from the measurement data transmitter 65 to a pulse detector 33 in the display device 1 illustrated in FIG. 1.

The respiratory measurement device 4 is configured to continuously measure respiration of a subject. The measurement data measured by the respiratory measurement device 4 is input to a respiratory cycle detection unit 41 of the display device 1.

The invasive blood pressure measurement device 5 is configured to measure a blood pressure by an invasive blood pressure (IBP) method, that is, by inserting a catheter into a blood vessel of a subject. The measurement data obtained by the invasive blood pressure measurement device 5 is input to an invasive blood pulse pressure measurement unit 51 of the display device 1.

The reception unit 6 is configured to receive an input operation of an operator, and configured to generate an instruction signal corresponding to the input operation. The reception unit 6 is, for example, a touch panel disposed to overlap the display 71 to be described later, an operation button provided on a casing of the display device 1, or a mouse or a keyboard connected to an input/output interface (for example, a USB interface) not illustrated. The instruction signal generated by the reception unit 6 is input to the display device 1.

<Configuration of Display Device>

The display device 1 can include the pulse detector 33, an A/D converter 34, the time interval detector 36, a calculator 70, a display controller 72, and a receiver 74. The calculator 70 can include the NIBP pulse pressure measurement unit 11, a heart rate calculation unit 12, a pulse wave transit time measurement unit 13, a pulse wave transit time variation measurement unit 14, a pulse amplitude measurement unit 15, a pulse amplitude variation measurement unit 16, a hemodynamic parameter calculation unit 17, an intrinsic coefficient calculation unit 18, a memory 19, the respiratory cycle detection unit 41, the invasive blood pulse pressure measurement unit 51, and a pulse pressure variation measurement unit 52. The display device 1 may include one or more central processing unit (CPU), a read only memory (ROM), a random-access memory (RAM), a hard disk drive (HDD), and the like. The CPU may function as the calculator 70, the display controller 72, and the receiver 74 and the like.

The time interval detector 36 is configured to obtain an electrocardiogram waveform, based on the measurement data received from the ECG electrodes 31 via the measurement data transmitter 65. The time interval detector 36 is configured to convert the measurement data into a digital signal, and is configured to output the digital signal to the heart rate calculation unit 12 and the pulse wave transit time measurement unit 13 of the calculator 70.

The pulse detector 33 is configured to obtain a waveform of the photoplethysmogram of the periphery, based on the measurement data received from the photoplethysmogram detection sensor 32 via the measurement data transmitter 65. Then, the pulse detector 33 is configured to output the measurement data to the A/D converter 34. The A/D converter 34 is configured to convert the measurement data into a digital signal, and is configured to output the digital signal to the pulse wave transit time measurement unit 13 and the pulse amplitude measurement unit 15 of the calculator 70.

The NIBP pulse pressure measurement unit 11 is configured to measure the NIBP pulse pressure, based on a blood pressure data measured by the blood pressure measurement device 2. The NIBP pulse pressure is calculated from a difference between a systolic (maximum) blood pressure value and a diastolic (minimum) blood pressure value. The measured NIBP pulse pressure is input to the intrinsic coefficient calculation unit 18.

The heart rate calculation unit 12 is configured to calculate the number of heartbeats (heart rate: HR) per minute, based on the reference point (R wave generation time point) measured by the time interval detector 36. The calculated heart rate HR is input to the hemodynamic parameter calculation unit 17.

The pulse wave transit time measurement unit 13 is configured to calculate a pulse wave transit time PWTT, which is an arrival time from an R wave to an SpO2 pulse wave of periphery in the electrocardiogram, based on the reference point (R wave generation time point) measured by the time interval detector 36 and the waveform of the periphery detected by the photoplethysmogram detection sensor 32.

More specifically, the pulse wave transit time measurement unit 13 is configured to calculate a moving average value of a plurality of pulse wave transit times immediately before a current time, in order to avoid disturbance of the value of the pulse wave transit time (PWTT) due to instantaneous noise or the like. Specifically, when calculating the pulse wave transit time PWTT consecutively 16 times, the pulse wave transit time measurement unit 13 is configured to calculate a moving average value of the 16 pulse wave transit times PWTT. Hereinafter, the moving average value of the 16 pulse wave transit times PWTT is referred to as a “moving average value PWTT-16”.

Further, when calculating a moving average value PWTT-16 consecutively four times using 64 consecutive pulse wave transit times PWTT, the pulse wave transit time measurement unit 13 is configured to calculate a moving average value of the four moving average values PWTT-16. The moving average value of the pulse wave transit time PWTT calculated in this manner is referred to as a “moving average value PWTT-64”. Then, the pulse wave transit time measurement unit 13 is configured to output the calculated moving average value PWTT-64, as the pulse wave transit time PWTT, to the hemodynamic parameter calculation unit 17 and the pulse wave transit time variation measurement unit 14.

The pulse wave transit time variation measurement unit 14 is configured to measure a respiratory variation of the pulse wave transit time PWTT, based on the pulse wave transit time PWTT calculated by the pulse wave transit time measurement unit 13 and based on a respiratory cycle detected by the respiratory cycle detection unit 41. Measurement data indicating the measured respiratory variation of the pulse wave transit time PWTT is input to the intrinsic coefficient calculation unit 18.

The pulse amplitude measurement unit 15 is configured to measure a pulse amplitude from the waveform of the extremities obtained by the pulse detector 33. The measured pulse amplitude is input to the pulse amplitude variation measurement unit 16.

The respiratory cycle detection unit 41 is configured to detect a respiratory cycle from the respiratory data measured by the respiratory measurement device 4. The detected respiration cycle is input to the pulse wave transit time variation measurement unit 14, the pulse amplitude variation measurement unit 16, and the pulse pressure variation measurement unit 52.

The pulse amplitude variation measurement unit 16 is configured to measure a pulse amplitude variation (PAV), based on the pulse amplitude measured by the pulse amplitude measurement unit 15 and based on the respiratory cycle detected by the respiratory cycle detection unit 41. The measured pulse amplitude variation is input to the intrinsic coefficient calculation unit 18.

The invasive blood pulse pressure measurement unit 51 is configured to measure an IBP pulse pressure, based on the blood pressure data measured by the invasive blood pressure measurement device 5. The measured IBP pulse pressure is input to the pulse pressure variation measurement unit 52.

The pulse pressure variation measurement unit 52 is configured to measure a pulse pressure variation (PPV), based on the IBP pulse pressure measured by the invasive blood pulse pressure measurement unit 51 and based on the respiratory cycle measured by the respiratory cycle detection unit 41. Measurement data indicating the measured pulse pressure variation is input to the intrinsic coefficient calculation unit 18.

The intrinsic coefficient calculation unit 18 is configured to calculate a coefficient intrinsic to a subject based on the NIBP pulse pressure measured by the NIBP pulse pressure measurement unit 11, the pulse wave transit time PWTT variation measured by the pulse wave transit time variation measurement unit 14, the pulse amplitude variation measured by the pulse amplitude variation measurement unit 16, and the pulse pressure variation measured by the pulse pressure variation measurement unit 52. The calculated coefficients are, for example, coefficients K, α, and β to be described later, and are input to the hemodynamic parameter calculation unit 17.

The hemodynamic parameter calculation unit 17 is configured to calculate the hemodynamic parameter of the subject, based on the heart rate HR calculated by the heart rate calculation unit 12, the pulse wave transit time PWTT measured by the pulse wave transit time measurement unit 13, and the coefficients K, α, and β calculated by the intrinsic coefficient calculation unit 18.

Here, it is assumed that the hemodynamic parameter calculation unit 17 is configured to calculate, as the hemodynamic parameters, a flow rate (stroke volume: SV) of blood flowing into a large artery during a systolic phase of a heart, and a cardiac output (estimated continuous cardiac output: esCCO) measured noninvasively and continuously.

Further, the hemodynamic parameter calculation unit 17 is configured to calculate a change rate of the hemodynamic parameter in a period (hereinafter, referred to as a “treatment period”) during which a treatment such as fluid administration to the subject or a treatment using a drug is performed. The calculation of the hemodynamic parameter and the calculation of the change rate of the hemodynamic parameter in the hemodynamic parameter calculation unit 17 will be described later.

<Calculation of Hemodynamic Parameter and Calculation of Change Rate of Hemodynamic Parameter>

(Calculation of Hemodynamic Parameter)

There is a correlation between stroke volume SV and pulse wave transit time PWTT as illustrated in Formula 1. In Formula 1, K, α, and β are coefficients intrinsic to a subject.

SV=K*(α*PWTT+β) (Formula 1)

The hemodynamic parameter calculation unit 17 is configured to substitute, into Formula 1, the coefficients K, α, and β calculated by the intrinsic coefficient calculation unit 18. For example, the hemodynamic parameter calculation unit 17 is configured to substitute, into the PWTT in Formula 1, the moving average value PWTT-64 received from the pulse wave transit time measurement unit 13. In this way, the hemodynamic parameter calculation unit 17 can calculate the stroke volume SV. The stroke volume SV calculated by the hemodynamic parameter calculation unit 17 is hereinafter referred to as “stroke volume esSV”.

The hemodynamic parameter calculation unit 17 is configured to periodically calculate the stroke volume esSV, and is configured to store, into the memory 19, the calculated stroke volume esSV and a calculation timing of the stroke volume esSV in association with each other, for example.

Further, in a case where an amount of blood (cardiac output: CO) driven by the beat of the heart is used, there is a correlation between stroke volume SV and heart rate HR as illustrated in Formula 2.

SV=CO/HR (Formula 2)

By using Formula 1 and Formula 2, estimated continuous cardiac output esCCO can be calculated as in the following Formula 3.

CO=SV*HR=

K*(α*PWTT+β)*HR

=esCCO (Formula 3)

The hemodynamic parameter calculation unit 17 is configured to substitute the coefficients K, α, and β into Formula 3. Then, the hemodynamic parameter calculation unit 17 is configured to substitute the moving average value PWTT-64 into the PWTT in Formula 3. Thus, the hemodynamic parameter calculation unit 17 can calculate the estimated continuous cardiac output esCCO.

The hemodynamic parameter calculation unit 17 is configured to periodically calculate the estimated continuous cardiac output esCCO, and is configured to store, into the memory 19, the calculated estimated continuous cardiac output esCCO and a calculation timing of the estimated continuous cardiac output esCCO in association with each other, for example.

(Calculation of Change Rate of Hemodynamic Parameter)

The operator who performs treatment on the subject can input a start timing of the treatment to the physiological information processing apparatus M by performing a predetermined input operation on the reception unit 6. In a case where such an input operation is performed, the reception unit 6 is configured to output, to the display device 1, a start signal indicating contents of the input operation and the start timing of the input operation.

The operator can input an end timing of the treatment to the physiological information processing apparatus M by performing a predetermined input operation on the reception unit 6. In a case where such an input operation is performed, the reception unit 6 is configured to output an end signal indicating an end timing to the display device 1.

When receiving the start signal or the end signal from the reception unit 6, the receiver 74 of the display device 1 is configured to output the received start signal or end signal to the calculator 70 and the display controller 72.

When receiving the start signal from the receiver 74, the hemodynamic parameter calculation unit 17 of the calculator 70 is configured to store the start timing indicated by the start signal into the memory 19. When receiving the end signal from the receiver 74, the hemodynamic parameter calculation unit 17 is configured to store the end timing indicated by the end signal into the memory 19.

After receiving the end signal, the hemodynamic parameter calculation unit 17 is configured to specify the treatment period, based on the start timing and the end timing. Then, the hemodynamic parameter calculation unit 17 is configured to calculate a change rate of the hemodynamic parameter in the specified treatment period.

For example, the hemodynamic parameter calculation unit 17 is configured to refer to the plurality of estimated continuous cardiac outputs esCCO stored in the memory 19, to specify a maximum value esCCOmax among the estimated continuous cardiac outputs esCCO calculated in the treatment period, and to specify a minimum value esCCOmin among the estimated continuous cardiac outputs esCCO calculated in the treatment period.

Then, by using the maximum value esCCOmax and the minimum value esCCOmin, the hemodynamic parameter calculation unit 17 is configured to calculate the change rate of the estimated continuous cardiac output esCCO as in the following Formula 4. The hemodynamic parameter calculation unit 17 is configured to store the calculated change rate into the memory 19.

Change Rate OF esCCO=2*(esCCOmax−esCCOmin)/(esCCOmax+esCCOmin) (Formula 4)

Further, the hemodynamic parameter calculation unit 17 is configured to refer to the plurality of stroke volumes esSV stored in the memory 19, to specify a maximum value esSVmax among the stroke volumes esSV calculated in the treatment period, and to specify a minimum value esSVmin among the stroke volumes esSV calculated in the treatment period.

Then, by using the maximum value esSVmax and the minimum value esSVmin, the hemodynamic parameter calculation unit 17 is configured to calculate the change rate of the stroke volume esSV as in the following Formula 5. The hemodynamic parameter calculation unit 17 is configured to store the calculated change rate into the memory 19.

Change Rate of esSV=2*(esSVmax−esSVmin)/(esSVmax+esSVmin) (Formula 5)

The start signal from the reception unit 6 may be a signal that does not include information on the start timing. In this case, for example, the receiver 74 is configured to notify the calculator 70 and the display controller 72 of the reception timing of the start signal as the start timing. Similarly or the same for the end signal, in a case where the end signal does not include information on the end timing, for example, the receiver 74 is configured to notify the calculator 70 and the display controller 72 of the reception timing of the end signal as the end timing.

<Display Control Processing>

(Display of Hemodynamic Parameter and Change Rate of Hemodynamic Parameter)

(a) Display During Period in which Treatment is not Performed

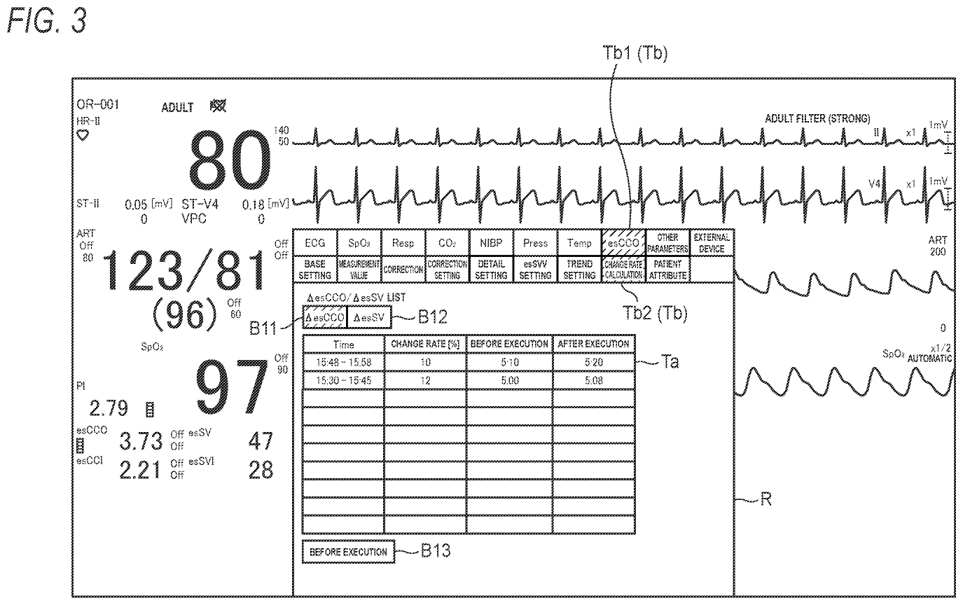

The display controller 72 is configured to perform control to output, to the display 71 that is a monitor or the like, the hemodynamic parameter and the change rate of the hemodynamic parameter calculated by the hemodynamic parameter calculation unit 17. Thus, the display 71 is configured to display a screen including the hemodynamic parameter and the change rate of the hemodynamic parameter. FIG. 3 illustrates an example of a screen displayed on the display 71 illustrated in FIG. 1.

As illustrated in FIG. 3, on the screen displayed on the display 71, a current heart rate, blood pressure value, estimated continuous cardiac output esCCO, stroke volume esSV, and the like of a subject are displayed. Further, the screen can include a region R in which the hemodynamic parameters in the treatment period of the subject are displayed.

The region R can include a plurality of tabs Tb. The plurality of tabs Tb can include, for example, a tab Tb1 for selecting a display related to the hemodynamic parameter, and a tab Tb2 for selecting a display related to the change rate of the hemodynamic parameter. For example, characters “esCCO” are attached to the tab Tb1. For example, characters “Change rate calculation” are attached to the tab Tb2.

FIG. 3 illustrates a screen displayed, during a period in which no treatment is performed on the subject, in a case where the operator selects the tab Tb1 and further performs an input operation of selecting the tab Tb2 on the reception unit 6 illustrated in FIG. 1.

In a case where the input operation as described above is performed, the reception unit 6 is configured to output, to the receiver 74 of the display device 1, an instruction signal indicating contents of the input operation. When receiving the instruction signal from the reception unit 6, the receiver 74 is configured to output the instruction signal to the display controller 72.

When receiving the instruction signal from the receiver 74, the display controller 72 is configured to read the start timing and the end timing stored in the memory 19, and to specify one or a plurality of treatment periods for the subject.

Further, the display controller 72 is configured to refer to the plurality of hemodynamic parameters and the plurality of change rates of the hemodynamic parameters stored in the memory 19, and to read, from the memory 19, for each treatment period, a start timing, an end timing, a hemodynamic parameter associated with the start timing, a hemodynamic parameter associated with the end timing, and a change rate of the hemodynamic parameter. Then, the display controller 72 is configured to perform control such that the read values are displayed in the region R.

Further, the display controller 72 is configured to switch the display of the change rate of the hemodynamic parameter between the display of the change rate of the estimated continuous cardiac output esCCO and the display of the change rate of the stroke volume esSV.

More specifically, the region R in a case where the tab Tb1 and the tab Tb2 are selected can include a selection button B11 for selecting the display of the estimated continuous cardiac output esCCO, a selection button B12 for selecting the display of the stroke volume esSV, and a table Ta illustrating a list of the change rate of the hemodynamic parameter for each treatment period. For example, characters “ΔesCCO” are attached to the selection button B11. For example, characters “ΔesSV” are attached to the selection button B12.

The operator can select any one of the selection button B11 and the selection button B12. In a state where neither the selection button B11 nor the selection button B12 is selected by the operator, the selection button B11 is automatically selected. That is, in such a state, the estimated continuous cardiac output esCCO and the change rate of the estimated continuous cardiac output esCCO are displayed in the table Ta.

Specifically, it is assumed that a first treatment is performed for the subject during a period from 15:30 to 15:45, and a second treatment is performed during a period from 15:48 to 15:58.

In this case, for example, it is displayed in the table Ta that the estimated continuous cardiac output esCCO at 15:30 is 5.00, the estimated continuous cardiac output esCCO at 15:45 is 5.08, and the change rate of the estimated continuous cardiac output esCCO in the first treatment period is 12%. Further, it is displayed in the table Ta that the estimated continuous cardiac output esCCO at 15:48 is 5.10, the estimated continuous cardiac output esCCO at 15:58 is 5.20, and the change rate of the estimated continuous cardiac output esCCO in the second treatment period is 10%.

In the table Ta, for example, a value for a latest treatment period is displayed in an upper row. Therefore, in a case where two treatments are performed, a value for the second treatment period is displayed in a first row, and a value for the first treatment period is displayed in a second row.

Further, it is assumed that the operator performs an input operation of selecting the selection button B12 included in the region R. In this case, the display controller 72 is configured to perform control such that the stroke volume esSV and the change rate of the stroke volume esSV for each treatment period are displayed in the table Ta, instead of the estimated continuous cardiac output esCCO and the change rate of the estimated continuous cardiac output esCCO for each treatment period.

The display controller 72 may be configured to perform control such that, in a case where a change rate that is less than a predetermined threshold exists among the change rates of the hemodynamic parameter displayed in the table Ta, the fact the change rate is less than the threshold is displayed in a manner of being easily recognized by the operator. For example, the display controller 72 may be configured to perform control such that a color of the change rate is displayed in a color different from colors of other change rates, or control such that a message indicating that the change rate is less than a threshold is displayed on the screen.

(b) Display During Treatment Period

The region R can further include a selection button B13 for inputting a start timing and an end timing of a treatment. In the selection button B13, for example, characters “Before execution” are attached in a period during which no treatment is performed, and characters “After execution” are attached during a treatment period.

As described above, the operator can input, to the physiological information processing apparatus M, the start timing and the end timing of the treatment, by performing a predetermined input operation on the reception unit 6. The predetermined input operation is, for example, an operation of selecting the selection button B13 displayed in the region R. That is, the operator performs an operation of selecting the selection button B13, to which the characters “Before execution” are attached, at the start timing of the treatment. Accordingly, the start timing is input to the physiological information processing apparatus M, and the characters on the selection button B13 are switched to “after execution”. Further, the operator performs an operation of selecting the selection button B13, to which the characters “After execution” are attached, at the end timing of the treatment. Accordingly, the end timing is input to the physiological information processing apparatus M, and the characters on the selection button B13 are switched to “before execution”.

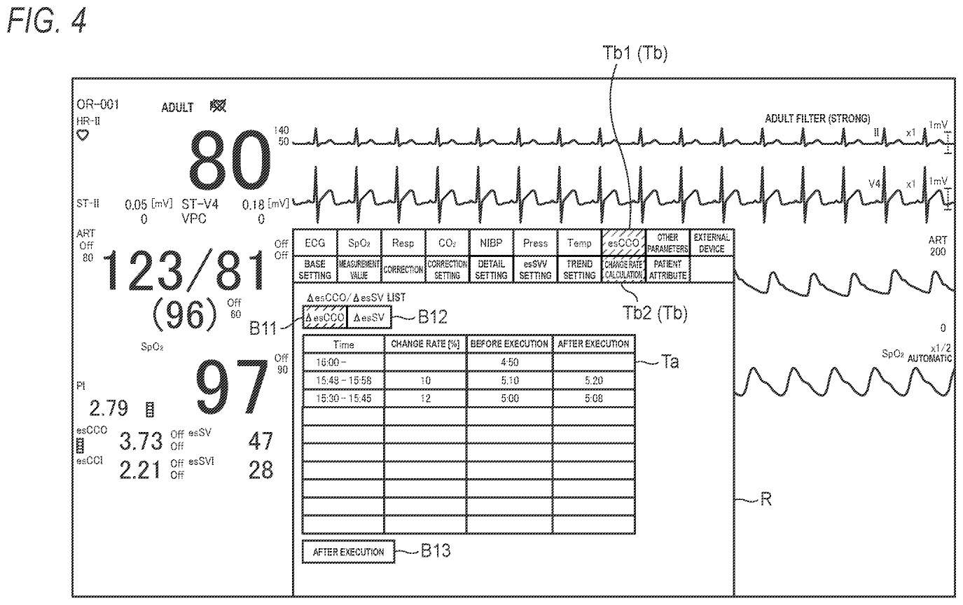

FIG. 4 illustrates an example of a screen displayed in a case where a measure for a subject is newly started. Referring to FIGS. 3 and 4, for example, it is assumed that the operator performs an input operation of selecting the selection button B13 illustrated in FIG. 3, on the reception unit 6 at 16:00 which is a timing at which a treatment for the subject is newly started.

In this case, the reception unit 6 is configured to output an instruction signal indicating the contents of the input operation and indicating a time of 16:00 that is the start timing, as a start signal, to the receiver 74 of the display device 1. When receiving the instruction signal output from the reception unit 6, the receiver 74 is configured to output the instruction signal to the calculator 70 and the display controller 72.

When receiving the instruction signal output from the receiver 74, the hemodynamic parameter calculation unit 17 in the calculator 70 is configured to store, into the memory 19 as the start timing, the time of 16:00 indicated by the instruction signal.

The display controller 72 is configured to perform control such that the characters attached to the selection button B13 on the screen are switched from “before execution” to “after execution” when receiving the instruction signal from the receiver 74. The display controller 72 is configured to refer to the plurality of hemodynamic parameters stored in the memory 19, and to read out, from the memory 19, the hemodynamic parameter corresponding to the time of 16:00, which is the start timing. Then, the display controller 72 is configured to perform control such that the start timing and the read value of the hemodynamic parameter are displayed in the table Ta.

Specifically, the display controller 72 is configured to perform control such that the time of 16:00 which is the start timing of the latest treatment and a value of the hemodynamic parameter at 16:00 are displayed in the first row of the table Ta. Further, the display controller 72 is configured to perform control such that each value for the treatment period from 15:48 to 15:58 displayed in the first row in FIG. 3 is displayed in the second row, and each value for the treatment period from 15:30 to 15:45 displayed in the second row in FIG. 3 is displayed in a third row.

(c) Display of Hemodynamic Parameter at Current Time

On the screen displayed on the display 71, as described above, the estimated continuous cardiac output esCCO and the stroke volume esSV of the subject at the current time are displayed. The display controller 72 is configured to periodically read the latest estimated continuous cardiac output esCCO and stroke volume esSV stored in the memory 19, and is configured to output, to the display 71, the read estimated continuous cardiac output esCCO and stroke volume esSV.

In the screen illustrated in FIG. 4, as an example, “3.73” that is the estimated continuous cardiac output esCCO at the current time and “47” that is the stroke volume esSV at the current time are displayed.

In the screens illustrated in FIGS. 3 and 4, both the estimated continuous cardiac output esCCO and the stroke volume esSV are displayed as the hemodynamic parameter of the subject at the current time, but any one of the estimated continuous cardiac output esCCO and the stroke volume esSV may be displayed.

(Switching of Moving Average Value of Pulse Wave Transit Time PWTT Used to Calculate Hemodynamic Parameter)

Here, in a case where the heart rate HR is 80 bpm, it takes about one minute to calculate the moving average value PWTT-64 of 64 pulse wave transit times PWTT. However, in a specific period such as a treatment period, it may be necessary to monitor a sudden hemodynamic change of the subject.

Therefore, for example, the physiological information processing apparatus M is configured to switch the moving average value of the pulse wave transit time PWTT used to calculate the hemodynamic parameter between the moving average value PWTT-64 of 64 pulse wave transit times PWTT (corresponding to a first pulse wave transit time) and the moving average value PWTT-16 of 16 pulse wave transit time PWTT (corresponding to a second pulse wave transit time).

(a) Automatic Switching

Referring again to FIG. 1, the pulse wave transit time measurement unit 13 is configured to output, to the hemodynamic parameter calculation unit 17, the moving average value PWTT-64 as the pulse wave transit time PWTT, during a period in which no treatment is performed, that is, during a period in which no start signal is received from the receiver 74. Accordingly, the hemodynamic parameter calculation unit 17 can calculate the hemodynamic parameter using the moving average value PWTT-64 with high accuracy in which the influence of noise or the like is reduced.

That is, during a period in which no treatment is performed, a value using the moving average value PWTT-64 is displayed on the display 71, as the current estimated continuous cardiac output esCCO and stroke volume esSV. Therefore, the operator can check more accurate changes in the estimated continuous cardiac output esCCO and the stroke volume esSV.

On the other hand, the pulse wave transit time measurement unit 13 is configured to output, to the hemodynamic parameter calculation unit 17, the moving average value PWTT-16 as the pulse wave transit time PWTT, during a treatment period, that is, a period in which the end signal is not received from the receiver 74 after the start signal is received from the receiver 74. Thus, the hemodynamic parameter calculation unit 17 can calculate the hemodynamic parameter using the moving average value PWTT-16 calculated at an early stage.

That is, in the treatment period, the current estimated continuous cardiac output esCCO and stroke volume esSV are frequently updated in the display 71. Therefore, the operator can quickly check the change in the estimated continuous cardiac output esCCO and the stroke volume esSV.

(b) Manual Switching

The pulse wave transit time measurement unit 13 may switch the moving average value, which is to be output to the hemodynamic parameter calculation unit 17 as the pulse wave transit time PWTT, between the moving average value PWTT-64 and the moving average value PWTT-16, in a case where a predetermined input operation is performed by the operator regardless of whether it is during the treatment period.

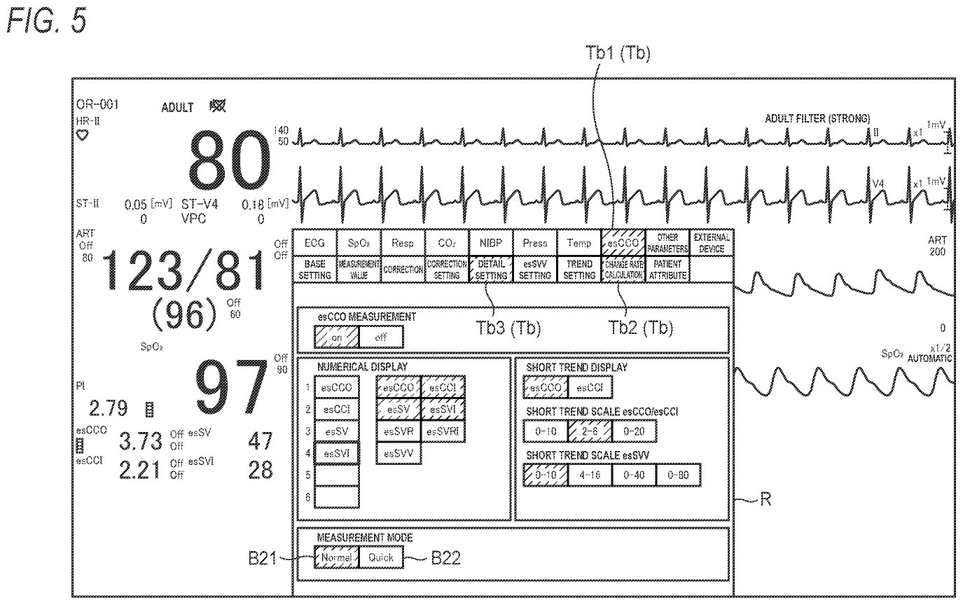

FIG. 5 illustrates an operation for instructing switching of the pulse wave transit time PWTT used to calculate the hemodynamic parameter. As illustrated in FIG. 5, the plurality of tabs Tb included in the region R can include a tab Tb3 for selecting display of a setting screen related to the hemodynamic parameter. For example, characters “Detail setting” are attached to the tab Tb3.

FIG. 5 illustrates a screen in a case where the operator performs an input operation of selecting the tab Tb1 and selecting the tab Tb3 on the reception unit 6 illustrated in FIG. 1. In a case where the input operation as described above is performed, the reception unit 6 is configured to output, to the receiver 74 of the display device 1, an instruction signal indicating contents of the input operation. When receiving the instruction signal from the reception unit 6, the receiver 74 is configured to output the instruction signal to the display controller 72.

When receiving the instruction signal from the receiver 74, the display controller 72 is configured to perform control based on the instruction signal such that a setting screen related to the hemodynamic parameter is displayed on the display 71 as illustrated in FIG. 5.

On the setting screen, selection buttons B21, B22 for selecting the pulse wave transit time PWTT used to calculate the hemodynamic parameter are displayed. For example, characters “Normal” are attached to the selection button B21. For example, characters “Quick” are attached to the selection button B22.

It is assumed that the operator performs an input operation of selecting the selection button B21 on the reception unit 6. In a case where the input operation as described above is performed, the reception unit 6 is configured to output, to the receiver 74 of the display device 1, an instruction signal indicating contents of the input operation. When receiving the instruction signal from the reception unit 6, the receiver 74 is configured to output the instruction signal to the calculator 70.

When receiving the instruction signal indicating that the selection button B21 is selected, the pulse wave transit time measurement unit 13 of the calculator 70 is configured to output, to the hemodynamic parameter calculation unit 17, the moving average value PWTT-64 as the pulse wave transit time PWTT. The hemodynamic parameter calculation unit 17 is configured to calculate the hemodynamic parameter using the moving average value PWTT-64.

Further, it is assumed that the operator performs an input operation of selecting the selection button B22 on the reception unit 6. In a case where the input operation as described above is performed, the reception unit 6 is configured to output, to the receiver 74 of the display device 1, an instruction signal indicating contents of the input operation. When receiving the instruction signal from the reception unit 6, the receiver 74 is configured to output the instruction signal to the calculator 70.

When receiving the instruction signal indicating that the selection button B22 is selected, the pulse wave transit time measurement unit 13 of the calculator 70 is configured to switch the moving average value, which is to be output to the hemodynamic parameter calculation unit 17 as the pulse wave transit time PWTT, from the moving average value PWTT-64 to the moving average value PWTT-16. Then, the hemodynamic parameter calculation unit 17 is configured to calculate the hemodynamic parameter using the moving average value PWTT-16.

In a state where neither the selection button B21 nor the selection button B22 is selected by the operator, the selection button B21 is automatically selected. Therefore, in such a state, the hemodynamic parameter calculation unit 17 is configured to calculate the hemodynamic parameter using the moving average value PWTT-64.

The moving average value of the pulse wave transit time PWTT that can be used to calculate the hemodynamic parameter is not limited to being switchable between two types of moving average value, which are the moving average value PWTT-16 and the moving average value PWTT-64, and may be switchable between three or more types of moving average values.

Further, even in a case where the tab Tb2 for selecting the display related to the change rate of the hemodynamic parameter is selected, the pulse wave transit time measurement unit 13 may be configured to switch the moving average value, which is to be output to the hemodynamic parameter calculation unit 17, from the moving average value PWTT-64 to the moving average value PWTT-16.

The physiological information display device including the hemodynamic parameter calculation unit 17, the intrinsic coefficient calculation unit 18, the memory 19, the display 71, the display controller 72, and the receiver 74 in the calculator 70 may be provided separately from the processing apparatus including other components of the calculator 70.

The display 71 may be provided inside the display device 1. In a case where the physiological information display device is provided separately from the processing apparatus as described above, the display 71 may be provided inside the physiological information display device.

<Flowchart of Operation>

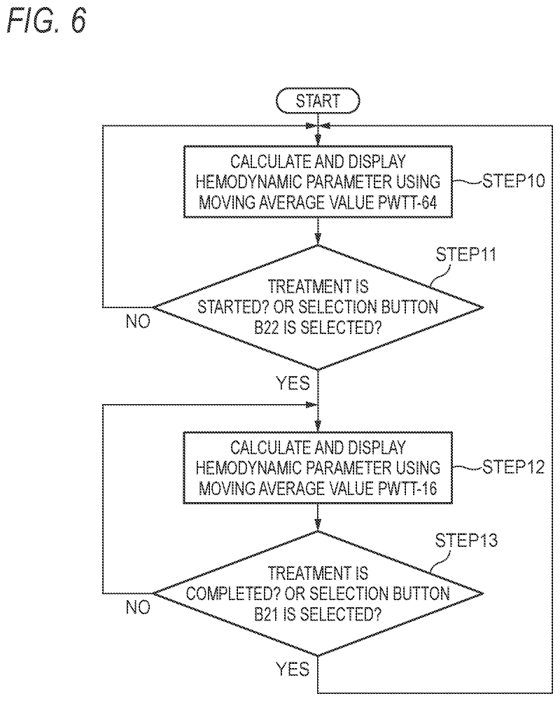

FIG. 6 is a flowchart illustrating the operation of switching the pulse wave transit time PWTT used to calculate the hemodynamic parameter in the physiological information processing apparatus M according to the embodiment of the presently disclosed subject matter.

With reference to FIG. 6, first, for example, in a case where the operator starts up the display device 1, each unit in the calculator 70 is configured to perform measurement or the like. At this time, the pulse wave transit time measurement unit 13 is configured to output, to the hemodynamic parameter calculation unit 17, the moving average value PWTT-64 as the pulse wave transit time PWTT, and the hemodynamic parameter calculation unit 17 is configured to calculate the hemodynamic parameter using the moving average value PWTT-64. Then, the hemodynamic parameter calculated by the hemodynamic parameter calculation unit 17 is displayed on the display 71 (STEP10).

Next, in a case where the treatment for the subject is not started, that is, in a case where the input operation of the start timing of the treatment has not been performed by the operator, or in a case where the operation of selecting the selection button B22 of FIG. 5 has not been performed by the operator (“NO” in STEP11), the operation illustrated in STEP10 is continuously performed.

On the other hand, in a case where the treatment for the subject is started, that is, in a case where the input operation of the start timing of the treatment has been performed by the operator, or in a case where the operation of selecting the selection button B22 of FIG. 5 has been performed by the operator (“YES” in STEP11). In this case, the pulse wave transit time measurement unit 13 is configured to switch the moving average value, which is to be output to the hemodynamic parameter calculation unit 17 as the pulse wave transit time PWTT, from the moving average value PWTT-64 to the moving average value PWTT-16. Then, the hemodynamic parameter calculation unit 17 is configured to calculate the hemodynamic parameter using the moving average value PWTT-16, and the display 71 is configured to display the hemodynamic parameter calculated by the hemodynamic parameter calculation unit 17 (STEP12).

Next, in a case where the treatment for the subject is completed, that is, in a case where the input operation of the end timing of the treatment has not been performed by the operator, or in a case where the operation of selecting the selection button B21 of FIG. 5 has not been performed by the operator (“NO” in STEP13), the operation illustrated in STEP12 is continuously performed.

On the other hand, in a case where the treatment for the subject is completed, that is, in a case where the input operation of the end timing of the treatment has been performed by the operator, or in a case where the operation of selecting the selection button B21 of FIG. has been performed by the operator (“YES” in STEP13), the operation illustrated in STEP is performed again. The operations from STEP 10 to STEP 13 are repeated, for example, until the operator stops the physiological information processing apparatus M.

As described above, in the physiological information processing apparatus M according to an aspect of the presently disclosed subject matter, the receiver 74 is configured to receive the start signal indicating the start timing of the treatment for the subject and the end signal indicating the end timing of the treatment. The calculator 70 is configured to calculate the moving average value of the pulse wave transit time PWTT of the subject, is configured to calculate the hemodynamic parameter of the subject using the calculated moving average value, and is further configured to calculate the change rate of the hemodynamic parameter in the treatment period from the start timing to the end timing, based on the start signal and the end signal received by the receiver 74. Then, the display controller 72 is configured to perform control to output the calculated change rate to the display 71.

As described above, with the configuration in which the change rate of the hemodynamic parameter is automatically calculated and displayed, the hemodynamic change of the subject during the treatment period for the subject can be visually and easily checked. In addition, by calculating the hemodynamic parameter using the moving average value of a plurality of pulse wave transit times, a more accurate value in which the influence of noise or the like is avoided can be displayed.

In the physiological information processing apparatus M according to another aspect of the presently disclosed subject matter, the display controller 72 is configured to switch the display of the change rate between the display of the change rate of the stroke volume (esSV) and the display of the change rate of the estimated continuous cardiac output (esCCO). According to such a configuration, the operator can freely select and check the change rate of the esSV and the change rate of the esCCO.

In the physiological information processing apparatus M according to another aspect of the presently disclosed subject matter, the display controller 72 is further configured to perform control such that the hemodynamic parameter calculated by the calculator 70 using the moving average value of a plurality of pulse wave transit times immediately before the current time is output to the display 71. With such a configuration, not only the change rate of the hemodynamic parameter during the treatment period but also the hemodynamic parameter can be quickly checked.

In the physiological information processing apparatus M according to another aspect of the presently disclosed subject matter, the display controller 72 is further configured to perform control of outputting, to the display 71, the start timing of the treatment, the end timing of the treatment, the hemodynamic parameter calculated using the moving average value of a plurality of pulse wave transit times immediately before the start timing, and the hemodynamic parameter calculated using the moving average value of a plurality of pulse wave transit times immediately before the end timing. With such a configuration, the hemodynamic parameters at the start timing and the end timing of the treatment for the subject can be checked.

In the physiological information processing apparatus M according to another aspect of the presently disclosed subject matter, the display controller 72 is configured to perform control of outputting, to the display 71, a list of change rates of a plurality of treatments, based on the start signal and the end signal received by the receiver 74. With such a configuration, the hemodynamic change in a plurality of treatments can be compared.

In the physiological information processing apparatus M according to another aspect of the presently disclosed subject matter, the calculator 70 is configured to calculate the moving average value of the pulse wave transit time PWTT of the subject, and is configured to calculate the hemodynamic parameter of the subject using the calculated moving average value. Further, the calculator 70 is configured to switch the calculation target between the hemodynamic parameter using the moving average value PWTT-64 of the first pulse wave transit time and the hemodynamic parameter using the moving average value PWTT-16 of the second pulse wave transit time that is shorter than the moving average time of the first pulse wave transit time.

With such a configuration, for example, in a situation in which a hemodynamic change of the subject should be checked early, the hemodynamic parameter is calculated using the moving average value PWTT-16 of the second pulse wave transit time, and in a situation in which a hemodynamic change with high accuracy in which the influence of noise or the like is reduced should be checked, the hemodynamic parameter can be calculated using the moving average value PWTT-64 of the first pulse wave transit time. Accordingly, the physiological information on the subject can be processed by a more appropriate method according to the use state.

In the physiological information processing apparatus M according to another aspect of the presently disclosed subject matter, the receiver 74 is configured to receive the start signal indicating the start timing of the treatment for a subject and the end signal indicating the end timing of the treatment. Based on the start signal and the end signal received by the receiver 74, the calculator 70 is configured to calculate the hemodynamic parameter using the moving average value PWTT-64 in a situation in which no treatment is performed, and is configured to calculate the hemodynamic parameter using the moving average value PWTT-16 during the treatment period.

Thus, during the period in which the treatment is performed on the subject, the hemodynamic change can be more quickly checked by calculating the hemodynamic parameter using the moving average value PWTT-16 of the second pulse wave transit time calculated early. On the other hand, in a situation where no treatment is performed, a more accurate hemodynamic change can be checked by calculating the hemodynamic parameter using the moving average value PWTT-64 corresponding to the first pulse wave transit time with high accuracy in which the influence of noise or the like is reduced.

In the physiological information processing apparatus M according to another aspect of the presently disclosed subject matter, the calculator 70 is configured to switch a calculation target from the hemodynamic parameter using the moving average value PWTT-64 to the hemodynamic parameter using the moving average value PWTT-16, in a case where the reception unit 6 for receiving an operation performed by the operator receives a predetermined operation. With such a configuration, the calculation target can be switched between the hemodynamic parameter using the moving average value PWTT-64 and the hemodynamic parameter using the moving average value PWTT-16 by the operator at any timing.

Although the embodiments of the present disclosure have been described above, the technical scope of the present application should not be construed as being limited to the description of the embodiments. The embodiments are merely an example, and it is understood by those skilled in the art that various modifications of the embodiments are possible within the scope of the inventions described in the claims. The technical scope of the present application should be determined based on the scope of the inventions described in the claims and equivalents thereof

What is claimed is:

1. A physiological information processing apparatus comprising:
a calculator configured to calculate:
a moving average value of a pulse wave transit time of a subject; and
a hemodynamic parameter of the subject, using the calculated moving average value,
wherein the calculator is configured to switch a calculation target between a first hemodynamic parameter and a second hemodynamic parameter, the first hemodynamic parameter being calculated using the moving average value of a first pulse wave transit time, the second hemodynamic parameter being calculated using the moving average value of a second pulse wave transit time that is shorter than the moving average time of the first pulse wave transit time.

2. The physiological information processing apparatus according to claim 1, further comprising:
a receiver configured to receive a start signal and an end signal, the start signal indicating a start timing of a treatment for the subject, the end signal indicating an end timing of the treatment,
wherein, based on the start signal and the end signal received by the receiver, the calculator is configured to calculate:
the first hemodynamic parameter, in a case where a treatment for the subject is not performed; and
the second hemodynamic parameter, in a case where a treatment for the subject is being performed.

3. The physiological information processing apparatus according to claim 1,
wherein, in a case where a reception unit receives a predetermined operation, the calculator is configured to switch the calculation target from the first hemodynamic parameter to the second hemodynamic parameter, the reception unit being configured to receive an operation performed by an operator.

4. A physiological information processing method comprising:
calculating:
a moving average value of a pulse wave transit time of a subject; and
a hemodynamic parameter of the subject, using the calculated moving average value,
wherein, in the calculating, a calculation target is switchable between a first hemodynamic parameter and a second hemodynamic parameter, the first hemodynamic parameter being calculated using the moving average value of a first pulse wave transit time, the second hemodynamic parameter being calculated using the moving average value of a second pulse wave transit time that is shorter than the moving average time of the first pulse wave transit time.
